(19) United States
(12) Patent Application Publication
(10) Pub. No.: US 2004/0133112 A1
(43) Pub. Date: Jul. 8, 2004

(54) System and method for macroscopic and confocal imaging of tissue

(76) Inventor: Milind Rajadhyaksha, Brookline, MA (US)

Correspondence Address: Kenneth J LuKacher, South Winton Court, Suite 204, 3136 Winton Road South, Rochester, NY, 14623, US

(21) Appl. No.: 10/471,332

(22) PCT Filed: Mar. 8, 2002
(86) PCT No.: PCT/US02/07173

(51) Int. Cl. A61B 6/00

(52) U.S. Cl. 600/476

(57) ABSTRACT

A system and method for imaging tissue samples is provided having a confocal microscope integrated with the confocal microscope. Such tissue samples may represent excised tissue from Moh's micrographic surgery. The system enables a user, such as a Moh's surgeon, to examine the low-resolution image of the tissue sample provided by the macroscope and identity sites that potentially appear to be abnormal, and then further examine the nuclear morphology of tissue at such sites in high-resolution images from the confocal microscope to detect the presence of abnormal tissue structures (cells), such as cancer. One or more agents such as acetic acid and calcein AM may be applied to the tissue sample to enhance the contrast of tissue structures in confocal and/or macroscopic images. Optics may be provided to enable the system to operate the macroscopic and confocal microscope in reflectance or fluorescence imaging modes.

### DESCRIPTION

This application claims the benefit of priority to U.S. Provisional Patent Application No. 60/274,887, filed Mar. 9, 2001.

### FIELD OF THE INVENTION

The present invention relates to system and method for macroscopic and confocal imaging of tissue, and especially for a system for macroscopic imaging integrated with a confocal imaging system for examination of tissue specimens, such as skin excisions obtained during Mohs micrographic surgery, using one or more contrast enhancement agents. The invention is especially suitable to enabling examination of tissue for abnormality, such as cancer, using low-resolution macroscopic imaging and high-resolution confocal imaging, thus avoiding histologic preparation of such tissue specimens. The system of the present invention may operate in reflectance or fluorescence imaging modes.

### BACKGROUND OF THE INVENTION

The removal of epithelial cancers in high-risk anatomical sites requires precise microsurgical excision with minimum damage to the surrounding normal tissue, and is guided by the histologic examination of each excision during the surgery. A well known example is Mohs micrographic surgery for excision of non-melanoma skin cancers. Non-melanoma skin cancers include basal- and squamous-cell cancers (BCCs, SCCs) that occur today at a rate of more than 1.2 million new cases every year, with treatment costs exceeding $500 million, as described in Marwick C., “New light on skin cancer mechanisms,” JAMA 1995; 275: 445-446. These cancers have high morbidity (physical and psychological trauma), occurring most frequently on the faces of people older than forty. Because the cancers occur in high-risk areas such as on or near the nose, eyes, ears, or mouth, precise microsurgical excision must be performed to remove only the cancer and leave the surrounding normal skin as intact as possible.

A Mohs procedure requires between one to several excisions, depending on the size, shape and complexity of the lesions. Frozen, hematoxylin and eosin (H&E)-stained, horizontal (en face) sections are prepared, to examine sub-surface superficial cancers (i.e., on and just below the surface of the excision). The processing for frozen sections requires 20-45 minutes for each excision during which the patient has to wait with an open wound under local anesthesia. Thus, a Mohs procedure typically lasts from one to several hours. This is slow and time-inefficient for Mohs surgeons, most of who perform several procedures per day.

Confocal microscopes enable high resolution optical imaging of tissue sections, thereby avoiding the preparation of frozen histology on slides. Such confocal microscopes can provide non-invasively images nuclear and cellular morphology in 2-5 μm thin sections in living human skin with lateral resolution of 0.5-1.0 μm. Examples of confocal microscopes or imaging systems are the VivaScope (™) manufactured by Lucid Inc. of Henrietta, N.Y. Other examples of confocal microscopes are described in U.S. Pat. Nos. 5,788,639, 5,880,880, and 5,719,700, published International Patent Application WO 96/21938, and in articles by Milind Rajadhyaksha et al., “In vivo Confocal Scanning Laser Microscopy of Human Skin: Melanin provides strong contrast,” The Journal of Investigative Dermatology, Volume 104, No. 6, June 1995, Milind Rajadhyaksha and James M. Zavislan, “Confocal laser microscope images tissue in vivo,” Laser Focus World, February 1997, pages 119-127, Rajadhyaksha et al., “In vivo confocal scanning laser microscopy of human skin II: Advances in instrumentation and comparison to histology,” J. Invest. Dermatol. 1999; 113: 293-303, Rajadhyaksha et al., “Video-rate confocal scanning laser microscope for imaging human tissues in vivo,” Appl. Opt. 1999; 38: 2105-2115, Masters et al., “Three-dimensional microscopic biopsy of in vivo human skin: a new technique based on a flexible confocal microscope,” J. Microsc. 1997; 185: 329-338, Masters et al., “Rapid observation of unfixed, unstained human skin biopsy specimens with confocal microscopy and visualization,” J. Biomed. Opt. 1997b; 2: 437-445, Corcuff et al., “In Vivo Vision of the Human Skin with the Tandem Scanning Microscope.” Dermatology 1993; 186: 50-54, Corcuff et al., “In vivo confocal microscopy of human skin: a new design for cosmetology and dermatology,” Scanning 1996; 18: 351-355, and New et al. “In Vivo Imaging of Human Teeth and Skin Using Real-Time Confocal Microscopy,” Scanning 1991; 13: 369-372. Further, optically sectioned microscopic images of tissue can be produced by optical coherence tomography or interferometry, such as described in Schmitt et al., “Optical characterization of disease tissues using low-coherence interferometry,” Proc. of SPIE, Volume 1889 (1993), or by a two-photon laser microscope, such as described in U.S. Pat. No. 5,034,613.

The confocal (optical) section thickness compares very well to the typically 5 μm-thin sections that are prepared for conventional (frozen or fixed) histology. Tissue morphology as well as dynamic processes can be imaged either in vivo or ex vivo (freshly excised) without any processing. It is a feature of the present invention to provide for confocal imaging of non-melanoma skin cancers during Mohs procedures without conventional histology. Rapid examination of the cancers within the skin excisions may be achieved within minutes. Confocal microscope can non-invasively optically image thin sections within turbid, scattering objects, without us having to physically cut the object into thin sections. Conventional microscopes cannot perform such optical sectioning, and require processing by physically cutting the object into thin sections with a microtome before viewing, i.e., histologic tissue preparation. However, confocal imaging has a serious limitation: the field-of-view is too small. With objective lenses of adequate numerical aperature or NA (0.3-0.9), the widest field-of-view is 1-2 mm. Mohs skin excisions are much larger (2-20 mm). Thus, it would desirable to provide an imaging system capable of providing low resolution images having a wide field of view of excised tissue and also high resolution confocal imaging of the excised tissue, which avoids the preparation of frozen histologically prepared excised tissue.

In examination of skin tissue for cancer in images of tissue sections from a confocal microscope, the dark spaces in-between collagen bundles in the dermis appear very similar to cancerous nuclei of atypical shapes and sizes. Nuclei in cancer cells are elongated, oriented and hence appear similar to the dark spaces in the dermis. Thus, when cancerous squamous (spinous) and basal cells from the epidermis invade the underlying dermis, as in BCCs and SCCs, optical detection of the cancer is difficult because the nuclei lack contrast relative to the surrounding normal collagen. It is therefore a further desirable feature to provide for contrast enhancement agents to tissue with an imaging system capable of low resolution imaging and high resolution confocal imaging of such tissue to make the cancer cells, often grouped in cancer nests, more detectable in imaged tissue.

### SUMMARY OF THE INVENTION

It is the principal feature of the present invention to provide an improved system for examination of tissue specimens by low resolution macroscopic imaging and high resolution confocal imaging similar to examination of traditional histological sections without requiring time-consuming and tedious preparation of traditionally histologic sections on slides.

It is another feature of the present invention to provide for an improved system for examination of tissue specimens by macroscopic low-resolution imaging and confocal high-resolution imaging using contrast agents applied to the tissue to enhance tissue structures.

Briefly described, the present invention embodies a system for imaging a tissue sample having a confocal microscope and a macroscope integrated with the confocal microscope. The confocal microscope has an objective lens through which scanned illumination is focused to the tissue sample and returned light is received from the tissue sample representing one or more confocal images of sections of the tissue sample. The macroscope includes a detector, such as a CCD (digital) camera, a light source coupled to a light guide for illuminating the tissue sample, optics for deflecting to the detector the light received from the tissue specimen through another objective lens disposed for imaging of the tissue sample, and optics for focusing the deflected light onto the detector. A holder is coupled to both the objective lens of the confocal microscope and the objective lens of the macroscope to enable selection of each of the objective lenses as needed for confocal and macroscopic imaging, respectively. One or more displays are provided for viewing images from the confocal microscope and the macroscope of the tissue sample. A programmed computer may be provided coupled to the displays for controlling the system and enabling user selection of one or both images from the confocal microscope and macroscope.

The present invention further provides a method useful for examination of tissue samples, especially when such samples represent excised tissue from Moh's micrographic surgery. The examination mimics the traditional Moh's surgeon procedure for examning histologically prepared tissue sections on slides by examining the low-resolution macroscopic image of the tissue sample on a display, identifying sites in the macroscopic image that potentially appear to be cancer nests, and examining the nuclear morphology in the high-resolution confocal images of the sites to detect the presence of abnormal tissue structures, such as cancer. By centering a site on a display of the low-resolution macroscopic image, the high-resolution confocal image corresponding to the site may be provided on the display, as such images are in registration with each other. The tissue sample may be moved, such as using a translation stage supporting the sample, to orient the tissue sample such that the site in the image is centered in the low-resolution macroscopic image. Alignment lines and/or a centered box may be provided in the low resolution macroscopic image to assist the user in locating a site at the center of the image.

One or more agents such as acetic acid and calcein AM may be applied to a tissue sample to enhance the contrast of tissue structures in confocal and/or macroscopic images. Optics may be provided to enable the system of the present invention to operate the macroscope and confocal microscope in reflectance or fluorescence imaging modes.

The term tissue sample is used herein to describe in-vivo tissue of the body of a patient, or the tissue of a patient surgically exposed either in-vivo or ex-vivo.

### BRIEF DESCRIPTION OF THE DRAWINGS

The foregoing features and advantages of the invention will become more apparent from a reading of the following description in connection with the accompanying drawings wherein,

FIG. 1 is a schematic diagram of the system according to the present invention in which a macroscope in integrated with a confocal microscope, such as the Vivascope (™) confocal microscope which is available from Lucid Inc. of Henrietta, N.Y. and is described in the above referenced U.S. Pat. No. 5,880,880;

FIG. 2 is a schematic diagram of the system of another embodiment according to the present invention in which a macroscope in integrated with a confocal microscope for enabling selection of different imaging modes, such as reflectance and fluorescence;

FIGS. 3A, 3B, 3C, and 3E are examples of confocal images of a tissue sample using acetic acid as a contrast enhancement agent, and FIGS. 3D and 3F show the corresponding traditional histological prepared samples of the tissue imaged in FIGS. 3C and 3E, respectively;

FIGS. 4A-4C are examples of macroscopic images of a tissue sample using acetic acid as a contrast enhancement agent;

FIGS. 5A-5C are examples of macroscopic images of a tissue sample using calcein AM as a contrast enhancement agent; and

FIG. 6 is an illustration of the process of examining tissue using the system of FIG. 1.

### DETAILED DESCRIPTION OF INVENTION

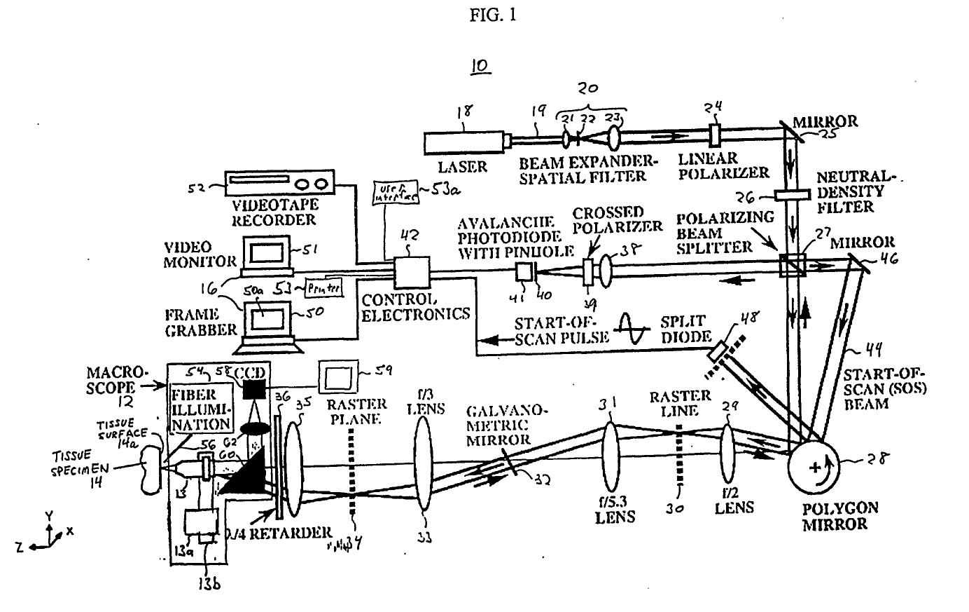

Referring to FIG. 1, a system 10 of the present invention is shown having a confocal microscope integrated with a macroscope 12. The confocal microscope is capable of producing one or more confocal images of sections of a tissue specimen or sample 14 on one or more displays 16. Tissue sample 14 may represent a skin excision, such as produced during Mohs micrographic surgery, or in-vivo tissue. The confocal microscope includes an objective lens 13 and all the elements in FIG. 1, but for such other elements (except lens 13) shown in the box labeled macroscope 12. A turret or holder 13b is provided holding objective lenses 13 and 13a to enable selection of imaging through one of the objective lenses in system 10, where objective lens 13 is used for confocal imaging and objective lens 13a is used for macroscopic imaging, as described in more detail below. A confocal microscope especially suitable in practicing the invention is described in U.S. Pat. No. 5,880,880, issued Mar. 9, 1999, which is herein incorporated by reference. Other confocal microscopes may also be used. In the confocal microscope, a laser or light source 18 produces a laser beam 19 through beam expander-spatial filter 20, which, for example, may be provided by a first lens 21 which narrows the beam and passes it through aperture 22 and then expands until collimated by lens 23. The beam from the expander-spatial filter 20 then passes through a linear polarizer 24, such as a half wave plate, and deflected by a mirror 25 through a neutral-density filter 26, a polarizing beam splitter 27, and then to a rotating polygon mirror 28. Neutral-density filter 26 may be, for example, a circular variable attenuator, such as manufactured by Newport Research Corporation. The beam is then deflected by polygon mirror 28 through lenses 29 and 31 onto a galvanometric mirror 32, which deflects the beam through lenses 33 and 35, a quarter wave plate 36 and objective lens 13 to tissue sample 14. The polygon mirror 28 and the galvanometric mirror 32 together produce the scanned beam which is focused by the objective lens 13 into a scanned focal spot through the tissue sample 14 under or on its surface 14a. The raster line 30 and raster plane 34 are illustrated in FIG. 1 by dashed lines to denote the angular scan of the beam in an x direction along a raster line generated by the rotation of polygon mirror 18, while the angular movement of galvanometric mirror 19 scans that raster line in a y direction orthogonal to the x direction to form a raster plane. For example, lens 29 may be an f/2 lens, lens 31 may be an f/5.3 lens, and lens 33 may be an f/3 lens. The laser beam 18 is of a wavelength or wavelength range which is transparent to the tissue to a desired depth from surface 14a. Objective lens 13 is preferably a dry objective lens, however a water immersion lens could also be used. Dry objective lenses are especially useful with examination of tissue excisions from Mohs surgery when only the top surface layers having superficial structures (such as 0 to 20 μm from top surface 14a) need be imaged.

The objective lens 13 collects reflected returned light from the tissue sample 14 to a detector 41 through quarter wave plate 36, lenses 33 and 35, galvanometric mirror 32, lenses 29 and 31, polygon mirror 28 to beam splitter 27. Beam splitter 27 deflects the returned light through a lens 38, a cross-polarizer 39, and a confocal aperture, such as a pinhole 40, to the detector 41, such as an avalanche photodiode. In this manner, a confocal image of a tissue section can be captured by control electronics 42 through detector 41. To provide a start of scan beam 44 to synchronize the control electronics 42 with the start of each raster line, the beam splitter 27 directs part of the beam incident the beam splitter 27 to rotating polygon mirror 28, via mirror 46, to a split diode 48 (e.g., photo-diode) which is connected to the control electronics 42 to provide a start of scan pulse at the beginning of each raster line. Confocal images are displayed on one of displays 16, which may represent a frame grabber 50 or video monitor 51, or the confocal image may be videotaped on videotape recorder 52, via control electronics 42. Two motors, not shown, one for each of mirrors 28 and 32, can provide the desired rotation and angular movement of respective mirrors 28 and 32. The speed of these motors may be controllable by control electronics 42. Control electronics 42 may represent a personal computer programmed to process the electronic signals from detector 41 into the raster of a confocal image in accordance with the position of the scanned beam along the raster line on the raster plane, and may use typical display driving software for outputting the confocal images on displays 16, or videotape recorder 52, or a printer 53, coupled to the computer. A user interface 53a, such as a keyboard or mouse, is provided to allow a user to control the operation of the system 10. Confocal images may provide nuclear and cellular detail of tissue sections at high resolution (such as sectioning of 2-5 μm) in small fields-of-view (such as 0.15-0.50 mm) to maximum possible depths (such as 200-350 μm), using longer near-infrared (800-1064 nm) wavelengths of laser beam 19.

The detected returned light on detector 41 is cross-polarized by cross-polarizer 39 with respect to the light polarized by linear polarizer 24, while the pinhole aperture 40 provides for spatially limiting the light of the return beam to a region of the tissue. Optionally, cross-polarizer 39 may be mounted on a rotatable stage to control the amount of polarization of the detected returned light such that detected light of the desired polarization other than crossed may be obtained. For example, cross-polarizer 39 may represent a linear polarizer.

Macroscope 12 images the tissue sample 14 at low resolution in a wide field of view, such as 2-8 mm. Turret 13b is moved to a position where the tissue sample 14 is imaged via objective lens 13a, rather than objective lens 13. Objective lens 13a has a lower magnification than lens 13, such as 2× to 10× to provide a field of view of 2 mm to 8 mm. For purposes of illustration, the figures of system 10 only show the position of turret 13b for imaging via objective lens 13. Although only two objective lenses are shown, turret 13b could have more than two objective lenses to provide a range of different magnifications. The macroscope 12 includes a bright lamp 54 coupled to a light guide 56, such as a optical fiber, for illuminating the tissue sample 14, and a CCD camera 58 which received light from objective lens 13a deflected by a beam splitter 60 and then focused by a lens 62 onto CCD camera 58. For purposes of illustration, only the CCD of this camera is shown. The light from lamp 54 illuminates the tissue sample at wavelengths, such as visible wavelengths, sensitive the CCD camera 58, and over an area corresponding to at least the desired field of view of the CCD camera. Accordingly, the light from fiber 56 penetrates the tissue to a depth. However, other illuminating wavelengths may be used in macroscope 12 with a corresponding sensitivity of the CCD camera to such wavelengths for other light penetration depth of the tissue sample 14 from surface 14a. The CCD camera 58 outputs a video signal, such as a typical digital camera, to a display monitor 59 representing a macroscopic (macro) image of tissue 14. However, this video signal, or a digital output of the macroscopic image, may be received by the control electronics 42, such that both the low resolution macroscopic image provided by the CCD camera and the high resolution confocal image provided by the confocal microscope are displayed simultaneously on one of displays 16, or are multiplexed such that the low and high resolution images switch with each other at a rate which cannot be perceived by the human eye. The user may select which of the images, confocal or macroscopic, to view on the display using user interface 53a, or to multiplex the images. Alternatively, the frame-grabber may have a separate input for the signal from the CCD camera 58.

Beam splitter 60 may be a large cube which deflects only the light collected by the objective lens 13a which is sensitive to the CCD camera 58 to the CCD camera. The illumination to the tissue sample and returned light from the tissue sample for the confocal microscope are not effected by the beam splitter 60, such that the macroscope does not interfere with imaging of the confocal microscope. In other words, the scan beam of the confocal microscope passes though beam splitter 60 to the tissue, and the returned light from the tissue of the confocal microscope, representing a section of the tissue, passes through the beam splitter from the tissue. Alternatively, the beam splitter 60 may be a plate oriented at 45 degrees parallel to the optical axis of lens 13a, which is large enough to allow passage of both the scan beam and the returned light of the confocal microscope.

The confocal microscope can provide a small field of view at the center of the macroscopic image from CCD camera 58. For the example, the field of view for a 20× objective lens 13a is about 1 mm. The light received from the tissue for macroscope 12 and the confocal microscope are both collinear, since the turret 13b aligns the objective lens 13a and 13, respectively, to have the same optical axis when each are positioned over tissue sample 14. Thus, the centers of the high and low resolution images are registered to each other. Thus, the high resolution confocal image of a horizontal section oriented parallel (enface) to the tissue surface 14 (parallel to an x,y plane) at a desired depth below surface 14a, correlates to the imaged tissue in the center of the low resolution macroscopic image. The specimen may be moved by a user to enable different areas of the tissue sample 14 to be examined in either imaging by the macroscope or confocal microscope. A stage (not shown) may be provided supporting the tissue sample to assist the user in moving the specimen in three orthogonal directions x,y,z. Optionally, the user may direct the computer of the control electronics to output the macroscopic image to the printer 53.

The macroscope 12 may similarly be integrated in other confocal microscopes, such as described in U.S. Pat. Nos. 5,788,639, 5,995,867, 6,134,009, 6,134,010, and 6,151,127, or other systems for imaging tissue sections using two-photon microscopy or optical coherence tomography. Optical coherence tomography or interferometry is described, for example, in Schmitt et al., “Optical characterization of disease tissues using low-coherence interferometry,” Proc. of SPIE, Volume 1889 (1993), while two-photon laser microscopy is describe, for example, in U.S. Pat. No. 5,034,613.

Optionally, linear polarizers and narrow (such as 10 nm) band-pass interference filters may be placed in the path of the light to the CCD camera 58. For example, illumination from lamp 54 via fiber 56 may be of low illumination power of 0.5-1.0 milliwatt on the tissue, and objective lens 13a may be a dry objective lens having a magnification of 2.5×-10× and numerical aperture (NA) 0.05-0.25 to provide a field of view of 2-8 mm. CCD camera 58 may be for example, a typical 500-pixel CCD camera, however a camera with larger or small number of pixels may also be used. The lens 62 directs the returned light into the aperture of the camera and onto one or more CCD array(s) of the camera. Preferably, the CCD camera provides gray scale imaging, but multiple color channel (such as RGB) may be used. The tissue sample 14 may be placed on a piece of gauze that is soaked with DPBS solution (to keep the tissue hydrated) under a standard 1 mm-thick microscope cover glass (to keep the tissue flat and still). However, other holding mechanisms may be used as cassettes described in U.S. patent applications Ser. Nos. 09/502,252 or 09/506,135, both filed on Feb. 17, 2000, having corresponding International Patent Application No. PCT/US00/04070 and PCT/US00/04128, respectively, or tissue sample holder contained in an enhancement solution bath as described in International Patent Application No. PCT/US00/07008, filed Mar. 17, 2000.

The system 10 operates the confocal microscope described above by reflectance imaging from the tissue sample. In another embodiment, the system 10 may operate by fluorescence imaging of the tissue sample in which illumination of the tissue sample 14 is of light at an excitation wavelength, and light is detected of the fluorescence wavelength. This may be achieved by replacing linear polarizer 24 with a bandpass filter to select an excitation wavelength of light, i.e., passing only the excitation wavelength and blocking all other wavelengths, when laser 18 provides beam 19 having multiple wavelengths or a range of one or more wavelengths, including the excitation wavelength. Alternatively, the linear polarizer 24 may be removed without replacement of a bandpass filter, and a laser 18 is provided which provides beam 19 at the excitation wavelength. Further, quarter wave plate 36 is removed, and the cross-polarizer 30 is replaced with a bandpass filter to select the fluorescence wavelength of light in the returned light from the tissue sample, i.e., passing the fluorescence wavelength and blocking the excitation wavelength. In the macroscope 12, a bandpass filter is located between the light guide 56 and the tissue surface 14a to select the exciting wavelength of the light from the light source 54 in the macroscope's illumination path, where the light source 54 produces light including the excitation wavelength, and another bandpass filter is located in front of the CCD camera 58 to select the florescence wavelength in the macroscope's detection path. Alternatively, instead of a bandpass filter in the macroscope's illumination path, a light source 54 is provided which provides light at the excitation wavelength.

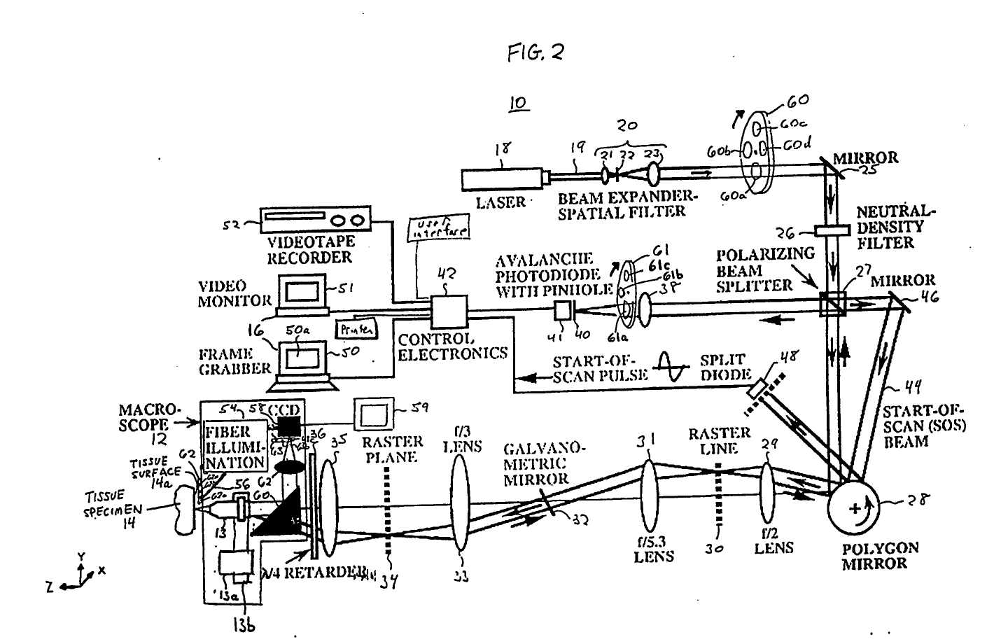

FIG. 2 shows the embodiment of system 10 enabling selection of different modes of operation of system 10 including reflectance and fluorescence imaging, in which both the confocal microscope and macroscope may operate in the same mode or in different modes. FIG. 2 is identical to FIG. 1, except that a wheel 60 replaces linear polarizer 24, a wheel 61 replaces cross-polarizer 39, a wheel 62 is located between the light guide 56 and the tissue surface 14a, and a wheel 63 is provided in front of the aperture of CCD camera 58 through which returned light for the macroscope is detected. Wheel 60 can be rotated to select one of multiple locations to provide along the illumination path of the confocal microscope one of a linear polarizer 60a, a bandpass filter 60b to select a single excitation wavelength from beam 19, or other polarizer or filter 60c, such as to provide circular polarization, or a short-pass bandpass filter 60d to select a range of wavelengths from beam 19. Wheel 61 can be rotated to select one of multiple locations to provide along the returned light path of the confocal microscope one of a cross-polarizer 61a, a bandpass filter 61b to select the fluorescence wavelength from returned light, and a long-pass filter 61c for selecting a range of wavelengths from the returned light. Wheel 62 is similar to wheel 60 in the illumination path of the macroscope 12, and includes an opening 62a, bandpass filter 62b to select a single excitation wavelength from light source 54, or a short-pass bandpass filter 62c to select a range of wavelengths from beam light source 54. Wheel 63 is similar to wheel 61 in the detection path of the macroscope, and includes an opening 63a, bandpass filter 63b to select the fluorescence wavelength in the detected light of the macroscope, and a long-pass filter 63c for selecting a range of wavelengths in the detected light of the macroscope. Wheels 60-63 may be similar to filter wheel used in optical microscopes, and may be manually positionable by a user, or automated by a motor coupled to the respective wheel, automatically rotated to a position by control electronics 42 coupled to such motors.

In reflectance imaging by the confocal macroscope, wheel 60 is positioned such that linear polarizer 60a is in the illumination path, and wheel 61 is positioned such that cross-polarizer 61a is in the returned light path. In reflectance imaging by the macroscope 12, wheel 62 is positioned such that opening 62a is in the illumination path, and wheel 63 is positioned such that opening 63a is in the detection path. In fluorescence imaging by the confocal microscope, wheel 60 is positioned such that bandpass filter 60b is in the illumination path, and wheel 61 is positioned such that bandpass filter 61b is in the returned light path. In fluorescence image by the macroscope 12, wheel 62 is positioned such that bandpass filter 62b is in the illumination path, and wheel 63 is positioned such that bandpass filter 63b is in the detection path. Other positions of wheels 60-63 may be used to provide other imaging characteristics as desired by the user.

Similarly, beam splitter 27 may be located on a wheel to enable selection of one of the polarized beam splitter 27 of FIG. 1, or other types of beam splitter, such as regular or nonpolarizing beam splitter, or a dichroic beam splitter. Such a dichroic beam splitter allows passage of the excitation wavelength in the illumination beam, and passage of the fluorescence wavelength in the returned light, and could be used instead of, or in combination with, bandpass filters in the illumination and return light path of the confocal microscope during fluorescence imaging. Similarly, such a beam splitter selection wheel could replace beam splitter 60 of the macroscope 12, to enable selection of one of beam splitter 60, or other types of beam splitters, such as a dichroic beam splitter which could be used instead of, or in combination with, bandpass filters in the illumination and detection path of the macroscope during fluorescence imaging.

To enhance the contrast of tissue structure in the low resolution macroscopic image provided by the macroscope and the high resolution confocal image provided by the confocal microscope in system 10, one or more agents may be applied to the tissue sample prior to imaging, such as acetic acid or calcein AM, or a combination of acetic acid and calcein AM.

In reflectance imaging, acetic acid causes whitening (acetowhitening) of epithelial tissue and makes the nuclei appear bright (instead of dark) in confocal images. This effect is described, for example, in Burghardt E. “Über die atypische Umwandlungszone,” Geburtsh. u. Frauenheilk. 1959; 19: 676; Smithpeter C, Dunn A, Drezek R, Collier T, Richards-Kortum R., “Near real-time confocal microscopy of cultured amelanotic cells: sources of signal, contrast agents and limits of contrast,” J. Biomed. Opt. 1998; 3: 429-436, and Drezek R A, Collier T, Brookner C K, Malpica A, Lotan R, Richards-Kortum R R, Follen M. “Laser scanning confocal microscopy of cervical tissue before and after application of acetic acid.” Am. J. Obstet. Gynecol. 2000; 182: 1135-1139. Acetic acid has the advantage that it is already in use by physicians to clinically differentiate dysplastic (abnormal) tissue versus surrounding normal tissue. For example, dermatologists use acetowhitening to observe genital warts and gynecologists to observe cervical dysplasia. The effect of acetowhitening, when normal and cancerous human skin may be washed with 5% acetic acid for three minutes, is excellently imaged in reflectance with the confocal microscope. However, other concentrations of acetic acid (such as 1-30%) and/or other wash duration may be used (such as 1 minute or less), may be used to provide the desired amount of brightening of the nuclei and cancer nests in confocal and macroscopic images. Citric acid has also been found to be a contrast enhancement agent, as described in U.S. patent application Ser. No. 60/241,092, filed Oct. 17, 2000, which is herein incorporated by reference. Multiple tissue samples may be imaged with system 10, such samples may represent excised tissue, such as between 2-20 mm large, but the number and size of the excised tissue depends on the particular Mohs surgery being performed.

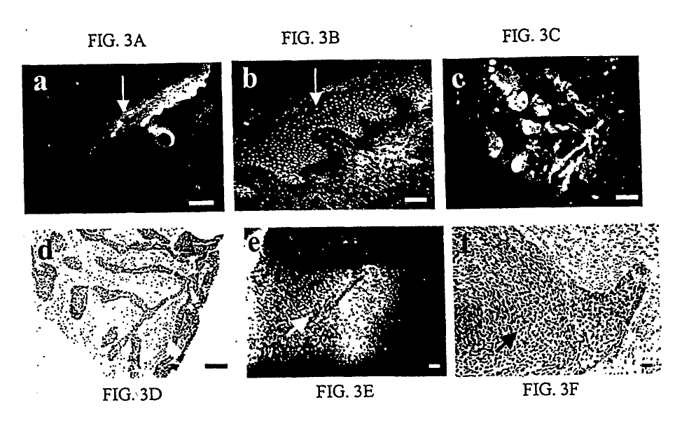

Examples of confocal images of normal human skin tissue after acetowhitening in reflectance imaging are shown in FIGS. 3A and 3B, where FIG. 3A shows brightening of the epidermis at confocal optics of 0.3 NA with a section thickness ≈30 μm, and FIG. 3B shows individual bright nuclei at higher resolution with confocal optics of 0.9 NA with section thickness ≈3 μm. In a BCC excision of tissue, bright nests of cancerous nuclei are shown in the confocal image of FIG. 3C (which has the same resolution as provided by confocal optics and section thickness of FIG. 3A), and correlates well to those seen in the corresponding traditional histological prepared sample of the same tissue in FIG. 3D. The atypical morphology of individual nuclei are shown in the confocal image of FIG. 3C (which has the same resolution as provided by confocal optics and section thickness of FIG. 3B), and correlates well to that seen in the corresponding traditional histological prepared sample of the same tissue of FIG. 3F. Scale bar is 25 μm in FIGS. 3A, 3B, 3E and 3F, and 100 μm in FIGS. 3C and 3D. The nuclei are believed to become bright due to acetic acid-induced condensation of chromatin. The contrast may be enhanced by imaging in crossed polarization in system 10, instead of brightfield, and detection of multiply back-scattered randomly polarized light from the intra-nuclear condensed chromatin, but suppress the singly back-scattered linearly polarized light from the surrounding dermis.

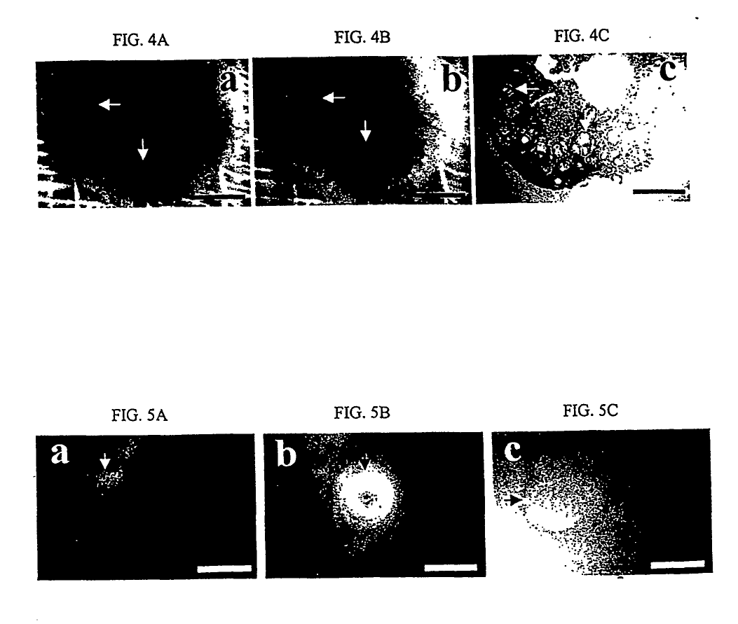

Examples of macroscopic images of normal human skin tissue after acetowhitening in reflectance imaging are shown in FIGS. 4A-4C, where FIGS. 4A and 4B show an acetowhitened BCC excision of tissue using a CCD camera and 2.5×/0.07 NA dry objective lens with a field-of-view of 8 mm. The illumination is limited to the sub-surface superficial cancer nests using violet 400 nm wavelength and crossed polarization (FIG. 4A). At a longer wavelength such as blue 488 nm that penetrates deeper, the contrast degrades due to back-scattered background. Cancer nests as well as normal skin structures in the macro-images (as indicated by arrows in FIGS. 4A, 4B) show good correlation to the corresponding histology (as indicated by arrows in FIG. 4C). Scale bar is 2 mm in FIGS. 4A-C.

As stated earlier, the confocal microscope and macroscope may be operated in fluorescence. In fluorescence, calcein AM is a viability dye that passively diffuse into cells and then labels cytoplasm in living cells. Calcein AM has an esterase substrate that, in living cells, is enzymatically cleaved to produce fluorescent calcein. Hence, calcein AM is believed to label the living cancer cells but not surrounding normal dermis. Another esterase substrate viability dye, fluorescein diacetate, may also be used as a contrast enhancement agent. Thus, a viability dye may be used to effectively label the cytoplasm in the living cancer cells and enhance the contrast of the cancer nests.

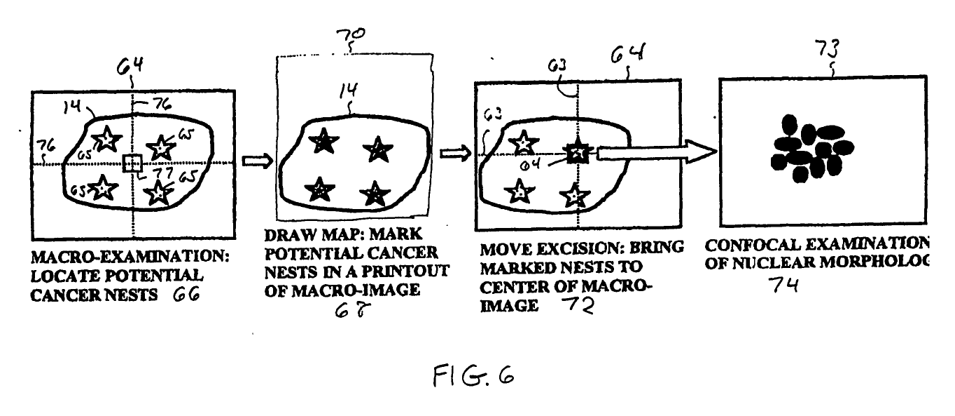

For example, macro-images of a fluorescein diacetate-labeled BCC excision using a CCD camera and 10×/0.25 NA dry objective lens with a field-of-view of 2 mm, in which the fluorescein diacetate forms fluorescein in living cells (at excitation wavelength of 488 nm, emission wavelength of 520 nm are shown in FIGS. 5A-5C. In FIG. 5A, dark nuclei (see arrow) within bright cytoplasm in the epidermis, while the underlying dermis is dark; in FIG. 5B bright hair follicle (see arrow) because it consists of living cells; and in FIG. 5C bright cancer nests (see arrow). The dermis adjacent to the hair follicle and cancer nests also appears bright rather than dark, because fluorescein may have leaked out of the cells. However, calcein AM is not leaky, and thus may be much more efficient for labeling only cancer nests but not the surrounding dermis. Scale bar is 0.5 mm in FIGS. 5A-5C.

Two contrast agents may be used with the tissue sample. Ideally, only one contrast agent is used (either acetic acid in reflectance or calcein AM in fluorescence) for both macro- and confocal imaging. There are, however, two possible limitations: (i) contrast in macro-reflectance images may not be strong enough, if the background noise from deeper tissue layers cannot be well suppressed, and (ii) signal-to-noise in confocal fluorescence images may not be strong enough, given that real-time high-resolution confocal detection may not be sensitive to fluorescence, assuming low, non-toxic concentrations of calcein AM. This, then, may necessitate the use of both contrast agents. In operation of system 10, macro-imaging may work best in fluorescence and confocal imaging in reflectance.

Macro-imaging of the macroscope integrated with confocal imaging provides both the patient and Mohs surgeon a faster (without frozen histology), more efficient examination of skin excisions during surgery, using the same range of resolution and magnification (field-of-view) as in conventional histology. Macro-imaging allows a user, such as a physician, to examine sub-surface superficial cancers nests at low resolution in wide fields-of-view. For example, in the low resolution macroscopic images from the macroscope 12 can detect and then examine the general morphology (i.e., shape, size, location) of sub-surface superficial cancer nests within large (2-20 mm) skin excisions. The user may then view confocal images to examine nuclear morphology in sub-surface superficial cancer nests at high resolution in small fields of view at such cancer nests found in the low resolution macroscopic picture. Confocal imaging enables the user to distinguish cancer nests from normal skin structures by examining their nuclear morphology. The macro-image delineates contrast-enhanced bright areas that are potentially cancer nests but, alternatively, they may be other commonly occurring normal skin structures such as hair follicles, sebaceous cells in sebaceous glands, or fat cells. These structures also consist of living cells, similar to cancer nests, and therefore will appear similarly bright due to the acetic acid or calcein AM. Distinguishing cancer nests from all the normal structures requires the Mohs surgeon to examine nuclear detail available in high resolution confocal images. In the cancer nests, the nuclei appear atypically enlarged, elongated, oriented and crowded; by comparison, the nuclei are small, circular and sparse in the normal structures. Within bright areas in the macro-images, such nuclear differences can be observed with the confocal images from the confocal microscope.

While macroscopic images provide low resolution imaging, confocal images provide nuclear and cellular detail at very high resolution (such as sectioning of 2-5 μm) in small fields-of-view (such as 0.15-0.50 mm) to maximum possible depths (such as 200-350 μm), using longer near-infrared (800-1064 nm) wavelengths of laser beam 19. As stated earlier a dry objective lens 13 is preferred, but a water immersion objective lenses may be used to minimize deep tissue-induced spherical aberrations. Objective lens 13 may have high numerical apertures (NAs) of 0.7-1.0. Small detector aperture (pinhole) 40 may have diameters of 1-5 resets.

In reflectance imaging using acetic acid, sub-surface superficial (maximum ˜100 μm deep) cancer nests with minimum background light from the deeper multiply-scattered light from deeper tissue may be imaged. Short violet/blue wavelengths illuminate only the superficial tissue layers since scattering prevents the light from penetrating too deep, as described in Anderson R R, Parrish J P. The optics of human skin. J. Invest. Dermatol. 1981; 77: 13-19, and Jacques S L, Roman J R, Lee K. Imaging of superficial tissues with polarized light. Lasers Surg. Med. 2000; 26: 119-129. Given the scattering and absorption coefficients at violet/blue wavelengths, the penetration depths in the dermis is believed to be limited to ≈8-12 scattering events for wavelengths 400-600 nm. In back-scatter, this corresponds to imaged depths of ≈4-6 scattering events, which is equivalent to ≈50-150 μm. Violet 400 nm wavelength light is thus the illumination wavelength of choice to image sub-surface superficial cancer nests to a depth of 50 μm. (50 μm corresponds to about ten histology sections, which is adequate for the Mohs surgeon.) Ultraviolet wavelengths 350-400 nm penetrate even less and would produce images of superficial cancer nests with better contrast. However, as ultraviolet light is phototoxic to skin, and we would not be able to use these wavelengths for intrasurgical imaging on patients. The contrast can be further enhanced with crossed polarizers. When tissue is illuminated with linearly polarized light, the specular reflection from the top surface retains its polarization, but the light that penetrates is randomly polarized, as described in the above cited Jacques et al. article, 2000, and in Demos S G, Alfano R R. Optical polarization imaging. Appl. Opt. 1997; 36: 150-155. The polarization is completely randomized in ≈10 scattering events that in back-scatter is, again, equivalent to a depth of ≈5 scattering events or ≈50 μm at 400 nm. Crossed polarization thus rejects the image from the top surface but improves visualization of sub-surface superficial structures (as in images in FIG. 4).

With violet 400 nm illumination and cross-polarized imaging, detection of nests in most skin excisions should be possible. In cases where the excision has very few nests or small nests including the potentially extreme case of a single small nest (This happens, for example, in sclerosing BCCs.) For such cases, image subtraction may be used (while keeping the illumination constant) to suppress the background and to further enhance the contrast and maximize detectability of the cancer nests on the very top tissue surface layer. The top surface of the skin excision is imaged with the short wavelength (λₛₕₒᵣₜ: 400 nm) and under parallel-polarized (par) condition; the deeper layers are imaged with long wavelength (λ_{long}: 450-500 nm) and under cross-polarized (cro) condition. Then, the following four types of image subtractions may provide images with strongly enhance contrast of the cancer nests on the top surface and suppress the deeper tissue background:

(i) Parallel-polarized subtract cross-polarized: I (par)−I (cro);

(ii) Short wavelength subtract longer wavelength: I (λₛₕₒᵣₜ)−I (λ_{long});

(iii) Short wavelength, parallel-polarized subtract long wavelength, cross-polarized: I (par, λₛₕₒᵣₜ)−I (cro, λ_{long});

and another option (that would be somewhat slower) is

(iv) After washing subtract before washing: I (after wash)−I (before wash).

Such image subtractions (i, ii, iii, or iv) may be provided by image processing in software by the personal computer of the control electronics 42, and the resulting image outputted to one or more of displays 16. Filtering of the light in the illumination and return/detection paths, as described earlier, and/or use of laser 18 or light source 54 may be used to provide the desired wavelength(s).

In fluorescence imaging using calcein AM, as mentioned before, calcein AM is believed to efficiently label the cytoplasm in the living cancer cells and enhance the contrast of the cancer nests without leaking out into the surrounding dermis. Consequently, there should be no background from the dermis. The excitation wavelength is 492 nm (488 nm band-pass filter for illumination) and the peak emission wavelength is 517 nm (520 nm band-pass filter for imaging). Light at 488 nm penetrates to a maximum depth of ≈200 μm. Illumination power of 0.5-1.0 milliwatt should be adequate. Similarly, filtering of the light in the illumination and return/detection paths, as described earlier, and/or use of laser 18 or light source 54 may be used to provide the desired wavelength(s).

Confocal imaging can examine nuclear morphology in sub-surface superficial cancer nests and nuclei at high resolution in small fields of view on or below the tissue surface 14a. For example, laser 18 may represent an argon-ion laser to produce beam 19 having blue 488 nm wavelength. Such illumination at 488 nm wavelength is useful for both reflectance and fluorescence imaging. Preferably, objective lens with low NA are used which give adequate sectioning to observe nuclear detail in confocal image. However, objective lens 13 with higher NA may also be used. Objective lenses with low NA also have low magnification, and hence provide a wider field of view than objective lens of higher NA. As stated earlier, objective lenses 16 may be a dry objective lens or a water immersion objective. For imaging excised tissue, immersion media (such as water or water-gels) may be used. However, when objective lens 13 is a dry objective lens, it may be used without an immersion media. One advantage of using a dry objective lens is the ability to rapidly change objective lenses without having to add or remove immersion medium.

The confocal section thickness is 1.4nλ/NA² where λ is the illumination wavelength and n is the immersion medium refractive index. To inspect nuclear detail, the confocal imaged section may be 5 μm thin. Thus, a dry objective of NA ≈0.35 may be used for which the magnification is ≈20× and field-of-view is ≈1 mm. This is equivalent to that used traditionally by the Mohs surgeon to examine histology. The objective turret holder 13b coupled to the objective lens 13 and 13a enable the macroscope and confocal microscope to use different lenses, as described earlier. For example, the confocal microscope provides a field-of-view of ≈1 mm when a 20× objective lens is used. To examine the entire skin excision, a two-dimensional sequence of confocal image images may be grabbed and aligned with each other using software, to create a composite confocal map. Using the map, nuclear morphology at any site (by looking at the individual images) may be provided. The sequence of images will be grabbed while moving the skin excision with a set of two-dirnensional (BY) stepper motor-driven translation stages coupled to the objective lens, which may automatically controlled by the computer of the control electronics. The stages will be driven using a typical step-grab-save software routine.

Referring to FIG. 6, the process of examination of a tissue sample 14 using system 10 is shown. First, a tissue sample 14, such as an excision for Moh's surgery, is placed on a translation stage under objective lens 13a. If the objective turret 13b is not already positioned for macroscopic imaging of the tissue sample 14, the user moves the turret to select imaging through lens 13a. One or more contrast enhancement agents are applied to the tissue sample 14 before or after placement on the stage. The surface of the tissue sample is planarized, if needed, such as by cover glass, or a transparent plate. A macroscopic image 64 of the tissue sample 14 from macroscope 12 appears on screen 50a of frame grabber 50 and is examined by a user, such as a Mohs surgeon. First, the user examines the low resolution, low-magnification (wide field of view) macroscopic image 64 of the tissue sample to identify sites 65 in the image that potentially appear to be cancer nests (step 66). The sites 65 are shown as stars for purposes of illustration. The features of interest to a Mohs surgeon when examining BCCs and SCCs in the low resolution macroscopic image 64 are nests of cancer cells, state of collagen and gross tissue architecture including normal structures such as hair follicles, sebaceous glands/cells fat cells and artifacts. To mark sites 65 which potentially appear to be cancer nests, the user may draw on a map on a print 70 of the macroscopic image 64 provided by printer 53 (step 68). One or all of the marked cancer nests at different sites 65 are brought to the center of the macroscopic image by moving the tissue sample 14 on its translation stage manually under the objective lens 13 (step 72). The user then moves the turret 13b (or the turret may be automatically moved by a motor coupled to the turret and connected to the control electronics 42) for imaging the tissue through objective lens 13, and then the nuclear morphology of cells in the high-resolution, high-magnification (small field of view) confocal image 73 are examined (step 74). Alignment lines 76 and/or a centered box 77 may be provided in the low-resolution macroscopic image 64 to assist the user in locating a site at the center of the image. Box 77 may be sized to approximately the field of view of the confocal image. In the high-resolution confocal image 73, the user examines nuclear morphology (i.e., the shape, size, orientation and density) to distinguish cancer (cell) nests from normal (healthy) structures. For each site, steps 72 and 74 are successively performed, in which images on screen 50a are switched by the user between macroscopic and confocal images by selecting images from detector 41 or 58 (CCD camera), respectively, with appropriate selection of objective lens 13a and 13, respectively. The macroscopic and confocal images are provided in real-time from the macroscope to the user. However, one or more of the macroscopic or confocal images may be stored in a memory or file on the computer of the control electronics and/or the video recorder, or printer. In the macroscopic image, attention can be paid to locate small or narrow nests that sometimes occur, as in sclerosing BCC's. Thus, system 10 can enable rapid examination of (non-melanoma) cancers in skin excisions during Mohs micrographic surgery, and such imaging will be similar to the traditional Mohs surgeon's procedure of examining histology sections. The locations of identified cancer in the tissue sample 14 are used as a guide for making additional excisions during the Mohs surgery, or to determine that the margins (boundaries) of the tissue sample are clear of cancer cells.

Real-time macro-imaging with a CCD camera integrated with confocal imaging offers a method to potentially avoid frozen histology and examine each skin excision within minutes. A combination of reflectance and fluorescence methods to enhance the contrast and detectability of the cancers may be used. Fast low-resolution examination of cancer nests in wide fields-of-view (macro-imaging) followed by high-resolution inspection of nuclear morphology in small fields-of-view (confocal imaging) can be performed in a manner that is similar to that for examining histology sections in resolution and magnification (i.e., field of view). Both the surgeon and the patient can potentially save several hours per day in the operating room. Real-time macro- and confocal-examination of excisions can improve the management of surgical pathology and guide microsurgery of any tissue.

From the foregoing description, it will be apparent that a system, method, and apparatus for macroscopic and confocal imaging tissue, and method for examination of tissue, have been provided. Variations and modifications in the herein described system, method, and apparatus in accordance with the invention will undoubtedly suggest themselves to those skilled in the art. Accordingly, the foregoing description should be taken as illustrative and not in a limiting sense.

1. An system for imaging a tissue sample comprising:
a confocal microscope having optics for providing one or more confocal images of sections of said tissue sample;
a macroscope integrated with said confocal microscope for providing an macroscopic images of said tissue sample having a larger field of view than said confocal images of said tissue sample; and
means coupled to said confocal microscope and macroscope for displaying said confocal and macroscopic images.

2. The system according to claim 1 wherein said confocal microscope has an objective lens through which scanned illumination is focused to said tissue sample and returned light received from said tissue in which said returned light represents one or more confocal images of sections of said tissue sample.

3. The system according to claim 2 wherein said macroscope comprises:
means for producing a macroscopic image of said tissue sample comprising a detector;
means for illuminating said tissue sample with light having wavelengths sensitive to said detector;
first optics for deflecting to said detector said light received from the tissue sample through another objective lens having wavelengths sensitive to said detector; and
second optics for focusing said deflected light onto said detector.

4. The system according to claim 1 wherein said confocal images and macroscopic images are registered with each other.

5. The system according to claim 1 wherein at least one image enhancement agent is applied to said tissue specimen to enhance tissue structures in said confocal and macroscopic images.

6. The system according to claim 5 wherein said image enhancement agent is one of acetic acid or calcein AM.

7. The system according to claim 5 wherein said image enhancement agent is acetic acid and calcein AM.

8. The system according to claim 3 wherein said detector is a CCD camera.

9. The system according to claim 3 wherein said objective lenses of said confocal microscope and macroscope each represents a dry objective lens, wherein said objective lens of said macroscope has a lower magnification than the objective lens of the confocal microscope.

10. The system according to claim 1 further comprising means for producing a confocal image representing the subtracting of a pair of confocal images taken at one of different wavelengths, different polarization states, different wavelengths and polarization states, or before and after application of an image enhancement agent.

11. The system according to claim 1 wherein said macroscope and confocal microscope have means for enabling one of reflectance and fluorescence imaging.

12. A method for imaging a tissue sample comprising the steps of:
imaging one or more images of sections of said tissue sample through an objective lens;
imaging one or more macroscopic images of said tissue sample having a larger field of view than said images of the section of said tissue sample; and
displaying said confocal and macroscopic images.

13. The method according to claim 12 comprising the step of applying an image enhancement agent to said tissue sample.

14. The method according to claim 12 wherein said image enhancement agent is one of acetic acid and calcein AM.

15. The method according to claim 12 wherein two of said images of section or two macroscopic images are subtracted from one another to enhance tissue structures.

16. The method according to claim 12 wherein said step of imaging sections is carried out by one of reflective and fluorescence imaging.

17. The method according to claim 12 wherein said step of imaging macroscopic images is carried out by one of reflective and fluorescence imaging.

18. A method for examining a tissue sample comprising the steps of:
providing a first image of the tissue sample in a first resolution;
selecting one or more locations in said first image having potentially abnormal tissue; and
providing a second image of the tissue sample at at least one of said locations having a higher resolution than said first resolution of said first image which is capable of diagnosing said tissue as having abnormal tissue.

19. An apparatus for imaging a tissue sample comprising:
first means for providing one or more images of sections of said tissue sample;
second means integrated with said first means for providing a macroscopic image of said tissue sample having a larger field of view than said images of said section of said tissue sample; and
a third means for displaying images produced by said first and second means.
